(19) United States
(12) Patent Application Publication
(10) Pub. No.: US 2026/0354940 A1
(43) Pub. Date: Nov. 12, 2026

(54) SELF-OSTEOTOME DENTAL IMPLANT AND ORTHOPEDIC FASTENER

(71) Applicant: SIMPLE IMPLANTS LTD, Tel Aviv (IL)

(72) Inventors: Oz Vachtenberg, Tel Aviv (IL); Ehud Teperovich, Ra'anana (IL)

(73) Assignee: SIMPLE IMPLANTS LTD, Tel Aviv (IL)

(21) Appl. No.: 19/129,377

(22) PCT Filed: Nov. 16, 2023
(86) PCT No.: PCT/IB2023/061606

§ 371 Date: May 13, 2025

Related U.S. Application Data
(60) Provisional application No. 63/426,121, filed on Nov. 17, 2022.

(51) Int. Cl. A61C 8/00

(52) U.S. Cl. CPC A61C 8/0024; A61C 8/0025; A61C 8/0092

(57) ABSTRACT

A self-osteotome dental implant includes an implant body having a coronal threaded region; and an apical region having a plurality of edgeless flutes. The implant is inserted into a bone in a low speed rotation of a dental implant and a simultaneous insertion of the implant into the bone.

### CROSS-REFERENCE TO RELATED PATENT APPLICATIONS

This application claims the benefit of provisional Application No. 63/426,121 entitled SELF-OSTEOTOME DENTAL IMPLANT AND ORTHOPEDIC FASTENER filed on Nov. 17, 2022.

### BACKGROUND

The present invention pertains to a dental implant and to an orthopedic fastener and implant and fastener placement procedures, specifically when the hosting bone is of poor quality.

In certain procedures conventional dental implants and orthopedic fasteners are secured to a bone in a multi-step process including creation of an osteotome with a first tool, densifying the bone surrounding the osteotome with a second tool and then securing an implant to bone.

Dental implants are used in the jaw while implants such as pedicle screws or orthopedic fasteners have been used to treat spinal disorders. The implants may also be secured to vertebra of the spine to secure rods extending between the vertebrae.

### SUMMARY

In one embodiment a method of placing a dental implant into a bone in a single rotational insertion of the dental implant includes: forming an osteotomy within a bone by a low speed rotation of a dental implant and a simultaneous insertion of the dental implant into the bone; densifying the bone by an edgeless shaped portion of the dental implant surrounding the osteotomy during the rotational insertion of the dental implant into the bone; and securing a coronal threaded portion of the dental implant to a coronal portion of the osteotomy at an end portion of the rotational insertion of the dental implant into the bone. A longitudinal axis of the coronal threaded portion is co-axial with a longitudinal axis of the osteotomy formed in the bone.

In one embodiment a self-osteotome dental implant includes an implant body having a coronal threaded region; and an apical region having a plurality of edgeless flutes.

In one embodiment a method of placing an orthopedic fastener into a bone in a single rotational insertion of the orthopedic fastener comprising: forming an osteotomy within a bone by a low speed rotation of an orthopedic fastener and a simultaneous insertion of the orthopedic fastener into the bone; densifying the bone surrounding the osteotomy during the rotational insertion of the orthopedic fastener into the bone; and securing a proximal threaded portion of the orthopedic fastener to a proximal portion of the osteotomy at an end portion of the rotational insertion of the orthopedic fastener into the bone.

In one embodiment an orthopedic fastener includes an implant body having a proximal threaded region; and a distal region having a plurality of edgeless flutes.

### BRIEF DESCRIPTION OF THE DRAWINGS

FIG. 1 is a plan view of an implant.

FIG. 1A is an isometric view of the implant of FIG. 1.

FIG. 2 is a cross sectional view of the implant of FIG. 1 taken along lines 2-2

FIG. 3A is a cross sectional view of the implant of FIG. 1, taken along lines 3A-3A.

FIG. 3B is a close up view of a portion of the implant of FIG. 3A.

FIG. 3C is a plan view of the implant of FIG. 1 taken from an apical end of the implant toward the coronal end.

FIG. 4 is an isometric view of a portion of the implant of FIG. 1.

FIG. 5 is a plan view of a single flute of the implant of FIG. 1.

FIG. 6 is an illustration of an insert prior to being inserted into a jawbone.

FIG. 7 is an illustration of the insert of FIG. 1 being inserted into the jawbone.

FIG. 8 is an illustration of the implant of FIG. 1 lifting a sinus floor.

FIG. 8A is an illustration of the implant of FIG. 1 in a first position with the fluted region being partially within the sinus cavity and illustrating displacement of bone.

FIG. 8B is an illustration of the implant of FIG. 8A shown turned 90 degrees.

FIG. 8C is a close up view of the apical portion of the implant in the orientation of the implant shown in FIG. 8.

FIG. 9 is an isometric view of an orthopedic fastener.

FIG. 10 is a distal isometric view of the orthopedic fastener of FIG. 9.

FIG. 11 is a plan view of the orthopedic fastener of FIG. 9.

FIG. 12 is a cross sectional view of the orthopedic fastener of FIG. 11.

FIG. 13 is a schematic view of a plurality of orthopedic fasteners in a portion of a spine.

FIG. 14 is a schematic partial cross-sectional view of the orthopedic fasteners in a portion of a spine.

FIG. 15 is an illustration of an implant orientated to be secured to a mandible.

FIG. 16 is an illustration of the implant of FIG. 15 secured to the mandible.

### DETAILED DESCRIPTION OF THE EXAMPLE EMBODIMENTS

Osseo integrated dental implants are typically metallic or ceramic screws that are placed in jawbone to support artificial teeth, after the loss of natural teeth. Although the majority of dental implantations are conducted with little to no complications, a few issues can arise that make the dental implant process more difficult, and in some cases, impossible. One such problem is when patients have a soft bone environment. Soft-quality bone lacks some of the properties that make it more resistant to micro-movements. When a bone environment is soft, the survival rate of the oral implant decreases. In one embodiment as described herein an implant and implant placement procedure works when the hosting bone is of poor quality. Soft bone typically means bone structure falling within type 3 or type 4 of the BQI (Bone Quality Index) known in the art.

Primary stability has a significant impact on the short and long-term success of dental implants. High primary stability can be achieved through implant geometry and its correlation to the bed preparation in the hosting bone.

Conventional implant bed preparation (osteotomy) involves the use of drills with increasing diameters; the final drill diameter is recommended by the implant company and is correlated to the shape and diameter of the implant being placed. In one embodiment the implant and implant procedures described herein provide an alternative to conventional drilling sequences to gain high primary stability in different clinical scenarios such as when placing implants into poor quality bone or when placing implants into extraction sockets.

In cases of very low-density bone (soft bone), bed preparation is attended to.

In such cases, under-drilling techniques and removing less bone from the site has been proven to be beneficial, by increasing initial bone-to-implant contact (BIC), by increasing primary stability through bone condensation around the implant, and by promoting overall bone healing. This can be achieved by using smaller diameter drills than in conventional protocols thus placing the implant into a smaller diameter osteotomy. Another alternative is to use an initial drill and then enlarge the site using osteotomes.

A bone-deforming instrument technique is generally a wedge-shaped tool which widens the bone gradually. Unlike drills, that take bone material out of the osteotomy, this technique allows for bone preservation and condensation.

In one prior art procedure osteotome preparation is done by a drilling sequence in a high-speed of 200 to 1200 RPM with irrigation while implant insertion described herein is done in a very low speed with no irrigation.

Several tools aiming to condense bone prior to implant placement are available on the market. These include the Densah® Burs, “screw shaped” expansion-condenser tools (Osteotomes BTLock) and hand instrument (Tatum's osteotomes). Typically, it is the clinician's decision as to how many drills to use, or which implant's shape and design to use with these tools.

Sinus lift procedure is a surgical procedure that is performed when there is not enough bone height in the upper jaw to support dental implant. This is accomplished by penetrating the sinus floor to the sinus cavity, lifting the sinus membrane gently for making a space to bone graft material.

Allograft, xenogeneic graft, and synthetic graft have been used as an additional bone substitute in a close sinus elevation technique.

Typical sinus lift procedures is a technique which includes at least two steps, first bone and sinus cavity preparations and then implant insertion.

Using prior art techniques, perforation of the Schneiderian membrane (the membranous lining of the maxillary sinus cavity) during a sinus lift operation, represents one of the more specific complications during sinus floor elevation.

Prior art techniques relate to bed preparation while in some cases, ignoring fixture shape and property, especially in sinus lift operation where a combination of soft bone and danger to the Schneiderian membrane are involved.

Prior art techniques relate to a surgical tool, resemble to conventional twisting drill. The shank is held by electrical power device, driven the prior art in a high speed. Due to the high speed, irrigation must be utilized, like with any conventional drill bit, for bone chips removal and heat reduction.

As described herein implant 110 has the properties of making the whole implantation process, which typically consists of multiple steps, into a single insertion step. As described herein in one continuous rotational insertion the osteotome is formed, the bone is densified, and the implant is secured to the bone. In one implementation the term continuous rotational insertion includes rotating implant 110 in a single direction such as clockwise without removing implant 110 from the bone. Continuous rotation may include rotation in a first direction, a pause in rotation, followed by further rotation in the first direction. In one implementation the first direction is clockwise rotation.

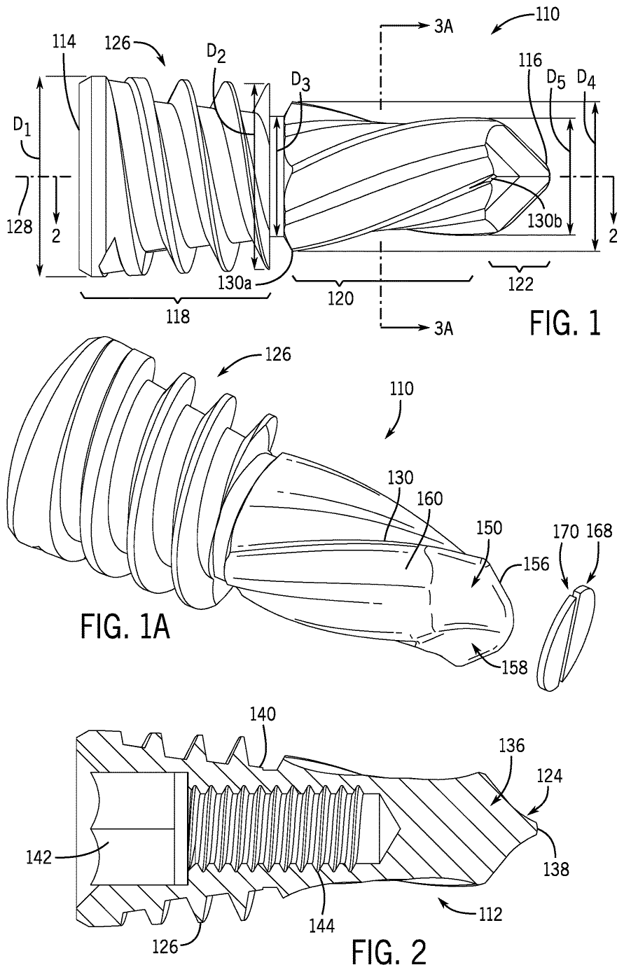

Referring to FIG. 1 a dental implant 110 includes an implant body 112 having a coronal end 114 and an opposing apical end 116. Implant body 112 includes a coronal region 118, and an apical region 120 between the coronal region and the apical end 116. A tip region 122 extends from the apical region 120 to the apical end 116. Tip region 122 includes a blunt tip 124.

The term apical end as used herein refers to a first end of the implant body 112 that located within a bone when implant body 112 is widening or creating the osteotome in a bone such as a jaw. The term coronal end as used herein refers to a second end of implant body 112 that is furthest from the apical end when the implant body 112 is secured to the bone.

Coronal region 118 includes a helical thread 126 tapered from a first major Diameter D1 proximate the coronal end to a second major diameter D2 proximate the distal or apical portion of helical thread 126. Referring to FIG. 4, helical thread 126 includes a minor diameter D3 at the leading edge 126a. Diameter, D3 is less than diameter D2 and diameter D2 is less than diameter D1. Diameters D1, D2 and D3 are measured perpendicular to a longitudinal axis 128 of implant body 112.

Apical region 120 includes a plurality of edgeless flutes 130. Each flute 130 helically extends about apical region 120 of implant body 112. In one implementation each flute 130 extends less than 360 degrees about implant body 112. Each flute includes a coronal end 130a and an opposing apical end 130b. Stated another way coronal end 130 a and apical end 130 b of each flute 130 is less than 360 degrees from one another. Each flute is defined by a pair of concave portions 130c and 130d and a convex portion 130e therebetween. The convex portion 130e is the portion that contacts the jawbone and densifies the bone as implant 110 is rotationally inserted into a bone of the jaw. Referring to FIG. 1 and FIG. 5, each flute 130 has a coronal end 130a and an apical end 130b. The difference in angular location of coronal end 130a and apical end 130b relative to longitudinal axis 128 is the amount that flute 130 extends about longitudinal axis 128. Referring to FIG. 5, in one implementation coronal end 130 a and apical end 130 b of flute 130 is approximately 90 degrees. The rotation of each flute about longitudinal axis 128 can vary based on the number of flutes. In one implementation with 3 flutes, the rotation about longitudinal axis 128 can be 120 degrees or between 0 degrees and 360 degrees. In one implementation the rotation about longitudinal axis 128 of each flute is greater than 360 degrees.

The shape and the geometry of apical region 120 is correlated to the shape and the size of helical thread 126. An outer diameter D4 of apical region 120 proximate the coronal end 134 is greater than the minor diameter D3 of helical thread 126. The outer diameter D4 of apical region 120 is twice the radius of the apex of convex portion 130e as measured from longitudinal axis 128 of implant body 112. Since diameter D4 of apical region 120 is greater than the minor diameter D3 of helical thread 126 helical thread 126 will first engage the bone below an opening of the osteotome. Measured from. Stated another way the bigger size of the tapered osteotome portion is slightly bigger than the minor thread diameter (root) and slightly smaller than the major thread diameter of the thread. The minor thread diameter goes beneath the entrance hole diameter made by the tapered shank for “catching” the bone and tapping its own thread into the bone.

Blunt tip 124 extends from an apical end of apical region 120 and is free of threads. Blunt tip 124 includes a body portion 136 terminating in a tip 138 that has a radius greater than 0.1 mm. In one implementation tip 138 has a radius greater than 0.15 mm. In one implementation tip 138 has a radius greater than 0.2 mm. In one implementation the radius of tip 138 is sufficient to not cut or grind the bone and not to cut the membrane in situation of sinus penetration as implant 110 is rotationally inserted into the bone.

Implant body 112 further includes a transition zone 140 between coronal region 118 and apical region 120. In one implementation transition zone 140 is free of threads and flutes. In one implementation transition zone 140 has a diameter that is the same as or less than the minor diameter D3 of helical thread 126.

Referring to FIG. 2 implant body 112 includes a recess 142 configured to receive an abutment to secure a prosthetic tooth to implant body 112. Recess 142 extends from an opening in coronal end 114 of implant body 112. A female threaded portion 144 extends from an apical bottom of recess 142 to threadedly receive a threaded portion of an abutment.

In one implementation a self-osteotome dental implant includes an implant body 112 having a coronal threaded region 118 and an apical region 120 having a plurality of edgeless flutes 130. Each edgeless flute 130 has a flute profile with a radius of curvature sufficient to densify a bone without grinding bone. In one implementation the radius of curvature of each flute profile is sufficient to densify the bone without cutting the bone. In one implementation each edgeless flute has a flute profile having a radius of curvature greater than 0.05 mm along an entirety of the flute profile. In one implementation each edgeless flute has a flute profile having a radius of curvature greater than 0.1 mm along an entirety of the flute profile. In one implementation each edgeless flute has a flute profile having a radius of curvature greater than 0.2 mm along an entirety of the flute profile. In one implementation each edgeless flute has a flute profile having a radius of curvature sufficient to densify the bone without cutting or grinding the bone when the dental implant 110 is rotationally inserted into the bone.

In one implementation each flute profile has a region of infinite curvature. The region of infinite curvature is the apex of the convex portion 130e of flute 130. The term edgeless as used herein means a transition on the flute and/or blunt tip 124 that does not cut or grind bone as implant 110 is being rotationally inserted into the bone of the jaw. In implementation each flute 130 has a first side and a second side. When implant 110 Is rotated clockwise about its longitudinal axis 128 second concave portion 130e is the leading side and concave portion 130c is the trailing side. In one implementation the profile of each flute 130 is edgeless along the entirety of convex portion 132e. In one implementation the profile of each flute 130 is edgeless along the entirety of convex portion 132e and first and second concave portions 130c, 130d. Even if implant 110 was rotated counterclockwise flutes 130 would not cut or grind bone as implant 110 is rotationally inserted into the bone. In one implementation flutes 130 does not cut or grind bone as implant 110 is rotationally inserted into the bone but rather densifies the bone. As a result, no bone is separated from the jaw as implant 110 is rotationally inserted into the bone of the jaw.

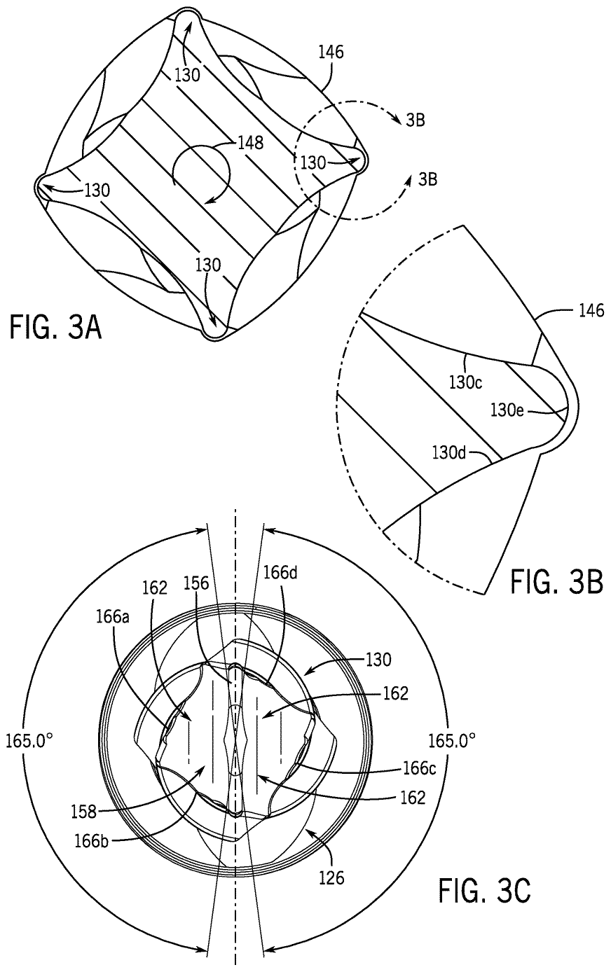

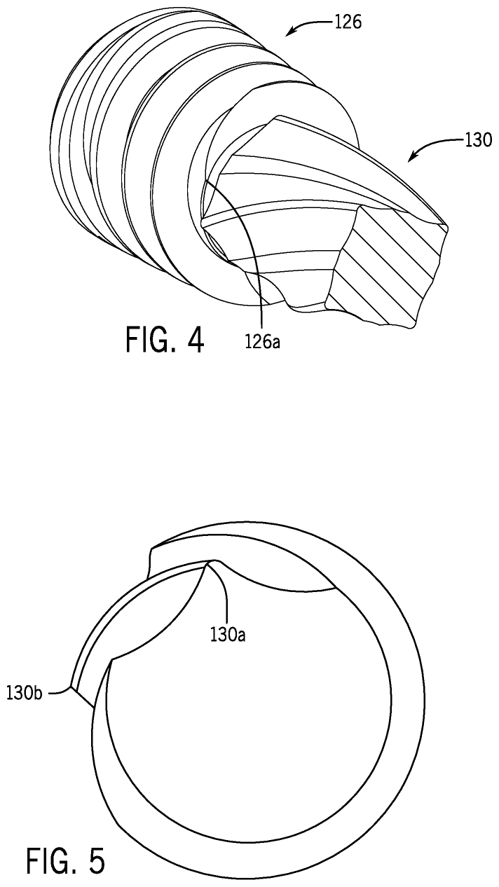

Referring to FIGS. 3A and 3B in one embodiment apical region 120 includes four flutes 130. However, it is contemplated that apical region 120 may include less than four flutes and in one implementation more than four flutes. FIG. 3A is a cross sectional representation of apical region 120 engaging with bone 146 of a jaw. Convex portion 130e of flute 130 engages bone 146 as implant 110 is rotationally inserted into bone 146. Convex portion 130e acts to densify bone 146 as the osteotome is enlarged from a first diameter D4 to a second diameter D5 as implant 110 is inserted into the bone. In one implementation implant 110 is rotated in a clockwise direction 148.

As noted above each edgeless flute extends less than 360 degrees about a longitudinal axis 128 of the implant body 112. In one implementation each flute extends between 0 degrees and 180 degrees about longitudinal axis 128. In one implementation each flute extends 45 degrees about longitudinal axis 128. Concave portion 130c of each flute 130 is adjunct a concave portion 130d of the adjacent flute 130. These concave portions 130c and 130d form a trough between the convex portions 130e of each flute. Stated another way the convex portion 130e forms an arcuate edgeless ridge of each flute. Where a trough is defined by the concave portions 132c, 132d of adjacent flutes separating the ridge defined by the convex portion 130e of adjacent edgeless flute 130.

In one implementation apical region 120 has a tapered shape between a first apical diameter D4 and a second apical diameter D5.

In one implementation the coronal threaded region 120 includes a thread 126 having a leading edge with a minor diameter D3 that is less than the first apical diameter D4. In one implementation, major diameter D2 of coronal thread 126 is greater than the first apical diameter D4.

In one implementation implant body 112 has a blunt tip 124 on an apical terminal end of the implant body 112. In one implementation the blunt tip has an edgeless arcuate shape greater than 0.1 mm. In one implementation the terminal portion of the blunt tip has a radius between 0.1 mm and 2. mm. In one implementation blunt tip 124 has a radius that is less than the diameter of the fluted region proximate the apical end portion of the convex portion of each flute 130.

In one implementation implant 110 includes a prosthesis located within an internal cavity 142 having an opening at a coronal end 114 of implant body 112. Note cavity and recess are used interchangeably herein.

In one implementation the apical region which widens the osteotome in a bone is made of bone integrated property material such as titanium or ceramic.

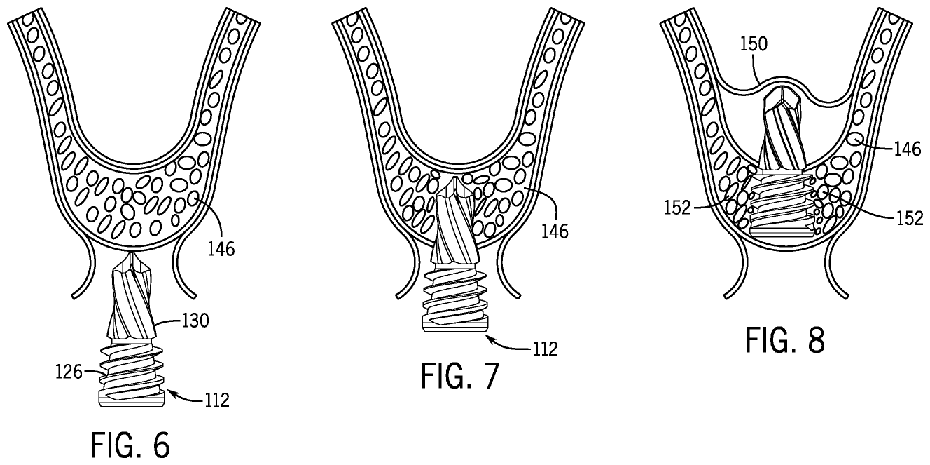

Referring to FIGS. 6-8 the method of placing a dental implant into a bone in a single rotational insertion of implant 110 includes forming an osteotomy within a bone by a low speed rotation of a dental implant and a simultaneous insertion of the dental implant into the bone; densifying the bone by an edgeless shaped portion of the dental implant surrounding the osteotomy during the rotational insertion of the dental implant into the bone; and securing a coronal threaded portion of the dental implant to a coronal portion of the osteotomy at an end portion of the rotational insertion of the dental implant into the bone.

In one implementation, blunt tip 124 first penetrates the bone in the jaw. The edgeless tip assists in providing a first densification of the bone, followed by the flutes 130 of apical region 120. Stated another way in one implementation the blunt tip 124 is pressed against the bone to form the osteotomy without first creating an osteotomy and osteotomy opening with a separate tool.

In one implementation dental implant 110 is rotated at a low speed of 50 revolutions per minute (rpm) or less. In one implementation implant 110 is rotated at a low speed between 10 and 25 revolutions per minute. The apical region 120 and the tip region 122 provide osteotome and osseodensification properties as implant 110 is rotationally inserted into the bone at a low speed. In one implementation rotation of dental implant 110 during the insertion is conducted manually free of a handheld powered rotational device.

Both the apical end 116 and the apical region 120 are formed with an edgeless feature to create osseodensification bed preparation. During implant 110 insertion the edgeless feature does not cut the bone nor make any debris. The curvature surface formed by a combination of concave and convex geometries provides for densifying the bone in a lateral direction by the apical region 120 and tip region 122. The result is to deform the bone structure and to ensure it remains intact. The lateral direction is perpendicular to the longitudinal axis 128 which is aligned with a longitudinal axis of the osteotomy being formed. Stated in another way, the general matrix of the bone remains intact as the bone is moved forward. In one implementation the bone matrix is the same or deformed but not fractured or broken.

Referring to FIG. 7, forming the osteotomy and densifying the bone occur during rotational insertion of the dental implant along a first linear depth. After which coronal region 118 begins to threadedly secure implant 110 to the bone. The osteotomy continues to be enlarged as apical region 120 is continuously inserted into the bone.

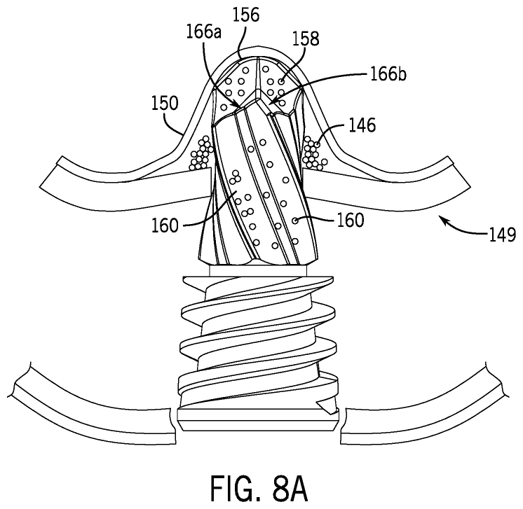

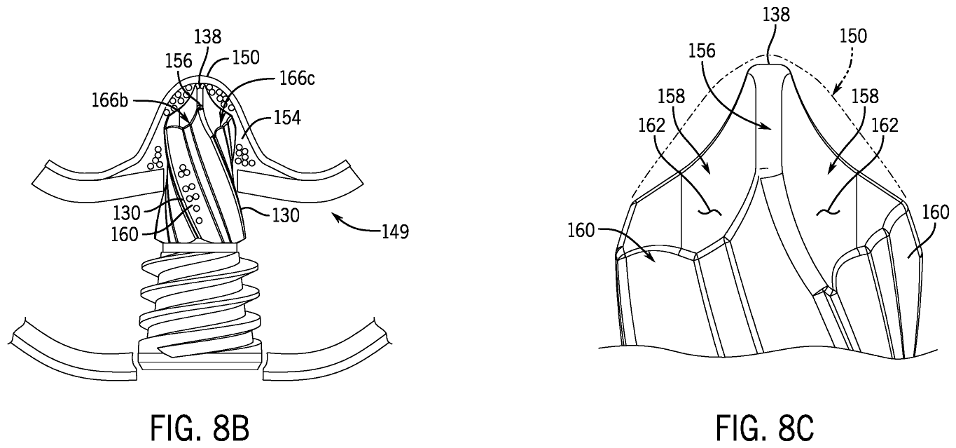

Referring to FIG. 8, in one implementation the method of placing implant 110 in the bone includes raising a sinus floor 149 with the blunt tip 124 during the rotational insertion of the dental implant into the bone. Blunt tip 124 enters through the sinus floor and lifts the Schneiderian membrane 150 into the sinus cavity. Schneiderian membrane 150 is not damaged during this process given the edgeless feature of the blunt tip 124.

Densifying the bone is performed by the plurality of edgeless flutes 130 on apical region 120 of the dental implant body 112. The osteotomy being created is enlarged during rotational insertion of implant body 112 into the bone of the jaw.

The bone is densified by the apical region 120 without cutting or grinding bone in any direction. The osteotomy is formed by gradually enlarging the osteotomy as the dental implant body 112 is rotationally inserted into the bone by a rotary sliding embossing process. Referring to FIG. 8, after insertion of implant 110 into the bone of a jaw the region 152 adjacent the helical thread 126 has been densified in comparison to the same region prior to insertion of implant 110 into the bone. Stated another way the region 152 of bone 146 adjacent implant 110 when implant 110 is fully secured to the bone is denser than the same region prior to the rotational insertion of implant 110 within the bone.

Referring to FIG. 8A, in one implementation the method of placing implant 110 in the bone includes raising Schneiderian membrane 150 adjacent the sinus floor with the blunt tip 124 during the rotational insertion of the dental implant into the bone. Blunt tip 124 enters through the sinus floor and lifts the gentle Schneiderian membrane 150 into the sinus cavity. Schneiderian membrane 150 is not damaged during this process given the edgeless feature of the blunt tip 124. A region 154 is formed between Schneiderian membrane 150 and bone 146. As is commonly understood in the art Schneiderian membrane 150 is the membranous lining of the maxillary sinus cavity. Blunt tip 124 includes an arcuate edgeless ridge 156 extending from a region adjacent the apical region 122 to the terminal most portion 138. In one implementation blunt tip 124 is completely edgeless. Tip region 122 is threadless between flutes 130 and the terminal most portion 138. In one implementation tip region 122 includes a second arcuate portion.

Densifying the bone is performed by the plurality of edgeless flutes 130 on apical region 120 of the dental implant body 112. In one implementation an osteotomy is created by insertion of the tip region 122 and apical region 120 and is enlarged during rotational insertion of implant body 112 into the bone of the jaw.

In one implementation as flutes 130 are moved into bone 146 of the jaw, a portion of the bone displaced by flutes 130 is densified and forced both outward and in an apical direction. Stated another way a portion of bone 146 displaced by implant 110 is densified in a radial direction that is perpendicular to the longitudinal axis of implant 110 and a portion of the bone 146 is displaced in an apical direction within the regions defined between each convex portion 130e of flutes 130. Stated yet another way a portion of the bone 146 is moved along the region between the convex portions 130 of each respective flute and moving into tip region 122. As the terminal end 138 of blunt tip 124 extends beyond bone 146 toward the sinus cavity the portion of bone 146 being displaced in the apical direction is moved into region 154. The term forward or forward direction as used herein will mean the direction from an opening in the bone into the bone along the longitudinal axis of the implant 110 as the implant is being engaged with the bone. Bone 146 that is defined as moving forward along regions 160 that follow the general spiral of flutes 130 since the bone that moves along regions 160 moves further from the opening of the jaw and coronal end of implant 110 toward the apical end of implant 110. In this manner a portion of bone 146 is moved forward from the region 152 in the jawbone into the region 154 of the sinus cavity. It is believed that under slow rotation of the implant, the bone mass in the jaw that includes trabecular structures that allows a portion of the cancellous bone mass to move toward and into the tip region 122 adjacent the blunt terminal end 138 with the bone structure generally intact. In contrast it is believed that high speed rotation of an implant acts to condense the bone of the jaw in substantial radial direction and any movement of bone in a direction generally along the longitudinal axis would not maintain the bone structure intact. Movement of bone along the longitudinal axis of the implant requires slow rotation of the implant in order for the bone structure to remain intact.

In one implementation the bone is densified and moved by the apical region 120 of implant 110 without cutting or grinding bone in any direction. Rather a portion of the bone in the jaw is moved by channeling and directing the flow of bone material to the tip region 122 to provide the bone material in region 154 between the jaw and Schneiderian membrane 150.

The osteotomy is formed by gradually enlarging the osteotomy as the dental implant body 112 is rotationally inserted into the bone by a rotary sliding embossing process. Referring to FIG. 8, after insertion of implant 110 into the bone of a jaw the region 152 adjacent helical thread 126 has been densified in comparison to the same region prior to insertion of implant 110 into the bone. Stated another way the region 152 of bone 146 adjacent implant 110 when implant 110 is fully secured to the bone is denser than the same region prior to the rotational insertion of implant 110 within the jaw.

Securing the coronal region 118 to a coronal portion of the osteotomy occurs during insertion of the dental implant to a second linear insertion depth. Helical thread 126 engages a wall of the osteotomy below a coronal opening of the osteotomy. Once implant body 112 is secured to the bone, an abutment is placed within an internal coronal portion of the dental implant. In one implementation the internal coronal portion includes a recess and a female threaded portion extending from the recess.

In one implementation forming the osteotomy is conducted free of irrigation. Stated another way irrigation with a fluid is not required during low speed formation of the osteotomy.

In one implementation the low speed rotation is set by a motor while the speed of linear insertion along the longitudinal axis of the osteotome is set by a force applied by the dentist along longitudinal axis. In one implantation longitudinal axis 128 is coaxial with the longitudinal axis of the osteotome.

In one implementation the placement of implant 110 requires an initial osteotomy similar to that required for every dental implant. In one implementation a pilot drill of between 2 mm-2.5 mm diameter is used to prepare an initial opening at the required location, to the required depth and angulation. Then, the apical segment of implant 110 paves the way to the threaded and the portion, condensing the soft bone as it inserted to position. The coronal segment of the implant, with its conical core thread, ensures gentle mechanical adherence to the surrounding bone and high primary stability.

Referring to FIG. 8, a sinus lift procedure is done when there is not enough bone height in the upper jaw while most of the time the remaining bone is poor quality. In one implementation an implant extends through the sinus floor and lifts the Schneiderian membrane 150 upward to make some room for additional bone. The space created under the implant-created-tent is filled with a blood clot rich in osteoblasts allowing for new bone formation. The edgeless blunt tip 124 has the capability to push the Schneiderian membrane 150 upward once it has been contacted. The edgeless curvature geometry of blunt tip 124 does not do any harm to the delicate tiny membrane attached to the sinus floor once they are in contact.

Referring to FIG. 1, FIG. 1A and FIG. 3A flutes130 helically extend in a counterclockwise direction about longitudinal axis 128. Stated another way apical end 130b of flute 130 is counterclockwise with respect to coronal end 130a of flute 130. In contrast helical thread 126 extends clockwise about longitudinal axis 128. The direction that implant 110 is rotated to place implant 110 in a bone is clockwise or the same direction as the rotation of helical thread 126 and opposite the direction of flutes 130. Stated another way the rotation direction of implant 110 is the same as the thread direction and opposite to the helical rotation of flutes 130. Referring to FIG. 1, threaded region includes a thread having a starting point adjacent the coronal end of the implant and terminating at a single distal point adjacent the apical region of the implant. In one embodiment there is only a single continuous thread in the coronal region extending from the starting point to the distal point. In one further implementation the single continuous thread extends about the longitudinal axis more than 720 degrees. Stated another way the single continuous thread is helical and extends about the implant body more than 2 times. In a further implementation the continuous thread extends about the implant body more than 4 times. In contrast, there are multiple flutes extending about the apical region of the implant, where each flute has a separate respective starting point and terminating at a respective distal point. Each flute extends about the apical region of the implant body less than 720 degrees. Stated another each flute is helical and extends about the implant body less than 2 times. In a further implementation each flute is spaced evenly apart from an adjacent flute and extends less than 1.5 times about the longitudinal axis of the apical region of the implant body. In a further implementation each flute extends one time or less times about the longitudinal axis of the apical region of the implant body. The direction and the angle the helix of the flute 130 created, defines the amount of bone which have been shifted radially and forwardly. In one implementation the angle of the helix of flutes 130 is 45 degrees. It is believed that a 45 degree angle of the helix of the flutes shifts 50% of the bone within the raceways forward and shifts 50% of the bone radially. As the angle of the helix of the flutes is reduced from 45 degrees more bone will be shifted radially than shifted forward. In one implementation the angle of the helix of flutes 130 is between 5 and 15 degrees. In one implementation the angle of the helix of flutes 130 is 10 degrees. In one helical thread 126 is a left winding thread and flute 130 is right winding. Stated another way helical thread 126 is a helical thread winding in a first direction and flutes 130 has a helical shape winding in a second direction opposite the first direction. It is contemplated that helical thread 126 winds right while flute 130 is helical and winds left. Left threads and right threads is a term well known in the art.

Referring to FIGS. 8A, 8B and 8C tip region 122 includes indentations 158 adjacent blunt tip 124 collectively covering more than 50% about the longitudinal axis 128 of the tip region 122. Stated another way indentations covers more than 50% of a leading surface area at the tip region of implant 110. Referring to FIG. 3C viewing the implant from an apical end toward the coronal end, the indentations cover more than 50% of the leading surface at the tip region. The leading surface area of the tip region has a peripheral outline defined by the indentations 158. Additionally, indentations 158 extend over 180 degrees about longitudinal axis 128. In one implementation tip region 122 includes indentations 158 adjacent blunt tip 124 collectively covering more than 90% (324 degrees) about the longitudinal axis 128 of the tip region 122. Stated another way indentations cover more than 90% of a leading surface area of implant 110. Referring to FIG. 3C, in one implementation indentations 158 extends 165 degrees about longitudinal axis 128 on both sides of arcuate ridge 156. In this implementation all of indentations 158 collectively extend 330 degrees about the longitudinal axis 128. In one implementation blunt tip 124 is formed of an arcuate ridge 156. In one implementation each end of arcuate ridge 156 extends from a respective flute 130. Each indentation 158 is in fluid communication with a region or raceway 160 between adjacent flutes 130. The bone material that is moved from the jaw along the region 160 between adjacent flutes 130 is deposited within indentations 158. Stated another way a volume of bone moved from the jaw to the region 154 between the floor of each cavity and Schneiderian membrane 150. In this manner a portion of bone from the jawbone of a patient is used to fill the gap between the implant and the Schneiderian membrane. As a result, there is no need to provide any other bone filler material. Between the implant and the Schneiderian membrane. The bone material covers more than fifty percent (50%) of the region between the Schneiderian membrane and a floor 162 of each indentation 158 covering a region that extends radially about a longitudinal axis of implant 110. In one implementation, the bone material moved from the jawbone to the region between the floor 162 of indentation 158 and the Schneiderian membrane coves substantially the entire of the region between the Schneiderian membrane and a floor of each cavity with the exception of the terminal end of arcuate ridge 156. In one implementation, the bone material covers more than ninety percent (90%) of the region between the Schneiderian membrane and a floor 162 of each indentation 158 covering a region that extends radially 360 degrees about a longitudinal axis of implant 110. Stated another way the bone material that is moved between flute 130 to the region between indentations 158 and the terminal end of arcuate ridge 156 extends radially 360 degrees about the longitudinal axis interrupted only by arcuate ridge 156. In the method of shifting bone material is discussed herein indentations 158 receive and contain bone shifted toward the apical end of the implant. In one implementation there are four indentations 158 each indentation 158 being separated from an adjacent indentation 158 by either arcuate ridge 156 or transition ridge 157. In one implementation each transition ridge 157 extends from an apical end of one flute 130 and extends to arcuate ridge 156. In one implementation transition ridge 157 extends toward arcuate ridge 156 but does not contact arcuate ridge 156. In one implementation transition ridge 157 does not exist but rather indentations 158 on either side of 156 are continuous. Stated another way bone material shifted from the raceways between adjacent flutes are deposited into a first indentation or cavity on one side of arcuate ridge 156 into a second indentation on the other side of arcuate ridge 156. Note that the bone material shifted to the indentations 158 is free to move on each side of arcuate ridge 156 without being constrained by a transition ridge 157. In one implementation indentation 158 has a concave shape sufficient to receive bone from the raceway between the flutes and deposit the bone in the sinus cavity between Schneiderian membrane 150 and the jawbone. Other shapes of each indentation 158 is contemplated such as a generally planar portions that provide an open cavity or open pocket that receives the bone from the raceways between adjacent flutes 130.

Referring to FIG. 8B, bone material is moved along a path 160 between adjacent flutes 130 into an indentation 158. Path 160 is also referred to herein as a raceway and a region. In one implementation there are four indentations 158 with each cavity being in fluid communication with a region 160. Referring to FIG. 8C a close-up of blunt tip 124 is shown at a 90 degree orientation to the view of blunt tip 124 in FIG. 8A. Arcuate ridge 156 in one implementation extends from a pair of flutes 130 and more specifically to a convex portion 130e of respective flutes 130. Arcuate ridge 156 has an arcuate shape as can be seen from FIG. 8A. In one implementation implant 110 includes four flutes 130 with a region 160 between each adjacent pair of flutes that are in fluid communication with at least one indentation 158. A portion of bone that is displaced from the jaw by rotation of implant 110 during forward insertion of implant 110 into the jaw moves along regions 160 and into indentations 158. As discussed herein when blunt tip 124 extends into the sinus cavity the portion of the bone that is displaced from the jaw along regions 160 and into indentations 158 is then deposited in the region 154 in the sinus cavity between Schneiderian membrane 150 and the jawbone. Referring to FIG. 8C, each region 160 includes a first concave portion 130c extending from a first convex flute portion 130e of one flute 130 and a second concave portion 130d extending from the second convex flute portion 130e of a second adjacent flute 130. In one implementation a portion 130f connects concave portion 130c and concave portion 130d that is non-concave. In one implementation portion 130f is concave such that the entire region between adjacent convex flute portions 130e is concave. In implementation concave portions 130c, concave portions 130d and portion 130f form define a continuous concave shape.

In one implementation indentations 158 that extend substantially 360 degrees about blunt tip 124 have a concave shape extending from the apical end 130b of flutes 130 such each indentation 158 can hold a portion of bone that is displaced from the jaw and moved along regions 160 is held between floor 162 of indentation 158 and Schneiderian membrane 150. Referring to FIG. 8C a silhouette of Schneiderian membrane 150 is shown. In one implementation arcuate ridge 156 is the only area of tip region 122 that is in contact with Schneiderian membrane 150. As illustrated in FIG. 8B the portion of bone that is deposited into indentations 158 covers the surface area of the tip region 122 between Schneiderian membrane 150 and the floors 162 of indentations 158. In one implementation a portion of the bone is shifted into the raceways and then moved along the raceways and deposited into the indentations 158 that are open faced such that a portion of the bone is located between the tip region and the bone in the case of a lower jaw or into the region between Schneiderian membrane 150 and the jawbone in the upper jaw. Since the tip region 122 is free of threads the portion of the bone shifted to the tip region 122 the bone portion forms a deposit substantially covering the entire apical end of the implant 110.

Referring to FIG. 3C tip region 122 includes indentations 158 that have a floor 162. Indentations 158 includes an outer periphery 166a, 166b, 166c and 166d. The portion of bone that is shifted along raceway 160 to indentations 158 fills the region extending from the outer periphery 166a, 166b, 166c, and 166d to the terminal end of arcuate ridge 156. Floor 162 of each indentation 158 and arcuate ridge 156 forms a pocket to hold a volume of bone that is shifted from the regions 160 between flutes 130 to tip region 122. The surface area of tip region 122 defined by floor 162 of indentations 158 from outer periphery 166a, 166b, 166c and 166d to arcuate ridge 156 is completely covered by the bone that is shifted from the jaw along regions 160 to indentations 158. Referring to FIGS. 8A and 8B the bone material fills the volume created between floor 162 and Schneiderian membrane 150 when implant 110 has raised the Schneiderian membrane 150. In one implementation bone 146 extends covers over 50% of the entire surface area of tip region 122 as viewed in a plane that perpendicular to longitudinal axis 128 of implant 110. The only portion of tip region 122 that is be covered by bone 146 is the terminal free end of arcuate ridge 156. Accordingly, in one implementation bone 146 covers over 90% of the entire surface area of tip region 122 as viewed in a plane that perpendicular to longitudinal axis 128 of implant 110. In one implementation, bone 146 between floor 162 of indentations 158 and Schneiderian membrane 150 forms a dome like volume only separated by arcuate ridge 156. Referring to FIG. 1A a surface area 168 of tip region is defined as the area perpendicular to the longitudinal axis of implant 110 that arcuate ridge 156 covers over one full rotation of implant 110. Note that bone material shifted from regions 160 to indentations 158 of tip region 122 covers the surface area 168 of tip region with the exception of an area 170 correlating to the width of arcuate ridge 156.

Referring to FIG. 1 implant 110 is shank free in that implant 110 has no shank portion attached to coronal region 118 that extends from coronal end 114 adjacent coronal region 118 in a direction away from the apical end 116. The helical direction of helical thread 126 is opposite the helical direction of flutes 130. Implant 110 is free of any threads in tip region 122 from apical region 120 to the terminal apical end of tip region 122. In the method of attachment of implant 110 to bone 146 described herein implant 110 is rotated slowly clockwise resulting in helical thread 126 engaging the sides of the osteotome while the oppositely helical direction of flutes 130 act to compress bone radially and displace bone forward along regions 160. In one implementation the method of attachment described herein results in implant 110 being secured within bone 146 free of the step of irrigating the osteotome during rotational insertion of implant 110 within bone 146. Rotational insertion is defined as rotating implant 110 clockwise while pushing implant 110 in a forward direction into bone 146. In one implementation, implant 110 is rotationally inserted into bone 146 without stopping to remove implant 110. In one implementation, implant 110 is inserted into bone 146 without a pumping motion in which implant 110 is moved fore and aft along the longitudinal axis of the osteotome which is coincident with the longitudinal axis 128. In one implementation all flutes 130 are edgeless and do not have a cutting edge when implant 110 is rotated clockwise or counterclockwise. In this manner flutes 130 displaces bone but does not cut or grind bone even if implant 110 is rotated both clockwise and counterclockwise during insertion of implant 110 within bone 146.

In one implementation implant 110 is continuously rotationally inserted into a bone such as a jawbone that does not include a prepared osteotome. Rotational insertion of implant 110 creates the osteotome and secures the implant 110 to the bone surrounding the osteotome in a single continuous step. Further in one implementation, bone is displaced from the jaw to a region in the sinus cavity between the lifted Schneiderian membrane 150 and the jawbone during the continuous insertion of implant 110 into the jawbone. In one implementation, only a portion of tip region 122 is positioned in the sinus cavity between Schneiderian membrane 150 and the sinus floor 149. In one implementation the entire tip region 122 is positioned in the sinus cavity between Schneiderian membrane 150 and sinus floor 149. In one implementation, in addition to the entire tip region 122 a portion of apical region 120 and flutes 130 is positioned in the sinus cavity between Schneiderian membrane 150 and sinus floor 149. In one implementation all of apical region 120 and flutes 130 is positioned within the region 154 between Schneiderian membrane 150 and sinus floor 149. In the case where at least a portion of the flutes extend into the sinus cavity, bone that is shifted along regions 160 and into indentations 158 is deposited in the sinus cavity between Schneiderian membrane 150 and sinus floor 149.

In one implementation a method of securing a dental implant includes securing the dental implant into the bone, surrounding tissue, peeling a floor of the sinus cavity and lifting the Schneiderian membrane. The term self-site defines all of the biological material that the implant contacts and interacts with during the method of securing a dental implant into a jaw of a patient.

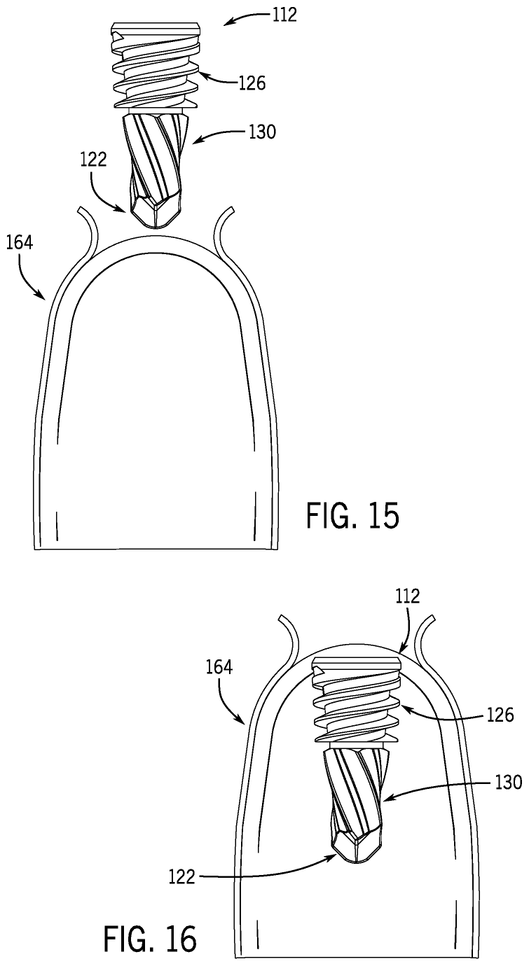

Referring to FIG. 15 and FIG. 16 the insertion of implant 110 includes creating rotatingly pushing implant body 112 into the bone of a patient's mandible 164 without a previous step of forming an osteotomy. A singular continual rotational insertion of implant 110 into the bone of mandible 164 acts to shift a portion of the bone in the mandible along regions 160 between flutes 130 to indentations 158 in tip region 122. A volume of bone is located between floor 162 and a hollow created by the rotation of arcuate ridge 156 when implant 110 is fully secured in s mandible 164. The operation of helical thread 126, flute 130. and tip region 122 operate in the same manner as discussed herein with respect to insertion of implant 110 in the upper jaw.

Rotary and straight thrust or pumping motion is one way prior art method that a surgeon uses a twist drill to develop an osteotomy to its full depth prior to implant insertion. During the formation of an osteotomy the surgeon needs to evaluate bone properties by the feedback the surgeon receives. In a situation where a sinus lift procedure is desired the dental implant undergoes a transition from soft bone in the jaw to harder bone tissue of the sinus floor. In such scenario, experience determines how much thrust to apply. In one prior art procedure, where the implant includes a thread on the apical end of the implant to move the implant forward inward in the jaw. However, when the bone in the jaw is soft and a thread portion on an apical end of the implant encounters a sinus floor that has bone that is harder than the bone in the jaw the implant body it is believed that the implant body might turn but without rectilinear motion leading to a loose grip of the entire dental implant within the bone. In one implementation implant 110 includes a single threaded region in the coronal portion of the dental implant and the apical region is free of a second threaded region. Stated another way implant 110 includes a single helical thread 126 at coronal region 118, a plurality of flutes 130 in apical region 120 and is free of a second helical thread between apical region 120 and the terminal apical free end in tip region 122. In one implementation a surgeon is able to vary the distance dental implant moves forward a first distance into the jaw per degree of rotation by varying the force and the feed applied to the dental implant. The surgeon is able to vary the distance the dental implant moves forward until the coronal thread 126 engages the interior walls of the osteotomy at which time the pitch of the thread dictates the distance dental implant moves forward per degree of rotation. In contrast, where the dental implant has a screw thread on the apical end of the dental implant the feed motion into the jaw is constrained by the pitch of the distal thread. In one implementation dental implant 110 only includes a thread 126 on the coronal portion of the implant body 112 and therefore the feed motion is free of constraint for a first distance equal to the distance between the terminal apical end of implant body 112 and the apical end of the coronal thread 126. Additionally, during the insertion of dental implant 110 for the first distance the surgeon may pull the dental implant 110 outward and reinsert the dental implant completely or partially since the apical end of the dental implant is thread free.

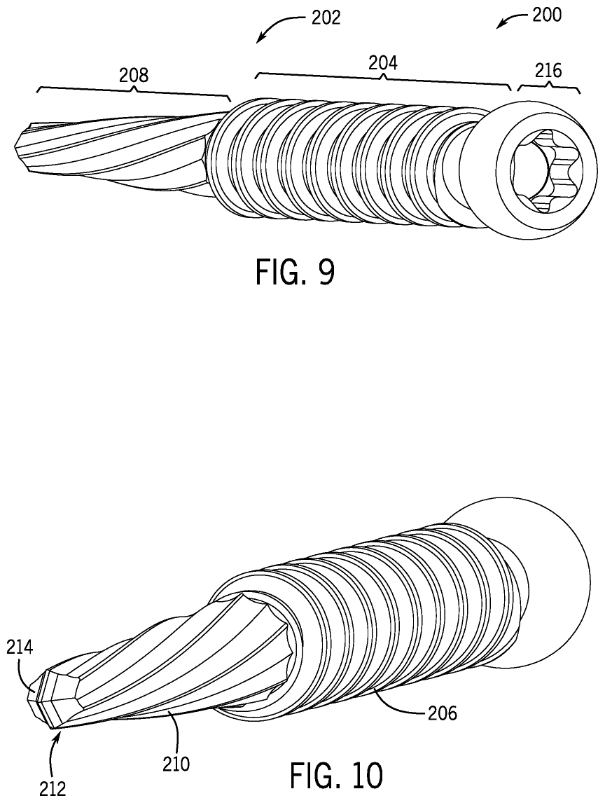

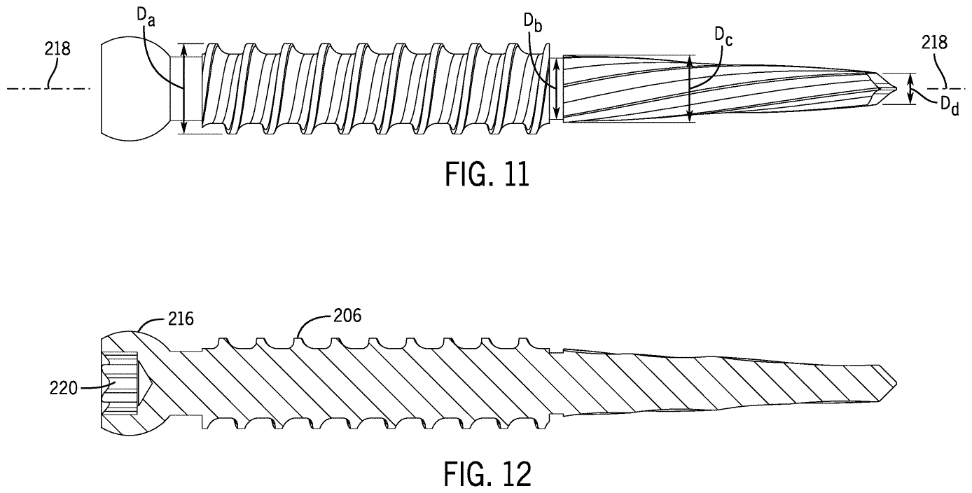

Referring to FIGS. 9-12 an orthopedic fastener 200 is secured to a vertebra (bone) of a patient. Bone screws for various spinal treatments and fixations are used in the art. Pedicle screws are used as bone anchoring elements to firmly grip the bone to facilitate attachment to the spinal implants. Using the pedicle screws' connection rod, surgeons can fixate the spinal segments together for spinal fusion. The pedicular fixation system (which consists of a minimum of four pedicle screws and two rods) can resist high loads and stabilize a fractured spine. Medical applications of pedicle screws show that tolerating the applied forces is possible for pedicle screws inside a healthy bone. When the bone is not healthy, poor screw fixation becomes a main concern.

Osteoporosis is a common bone disease in which the bone mineral density (BMD) is reduced. Osteoporosis decreases the bone strength which causes an increased risk of fracture in the bony structures of the patients. This disease is common in elderly people and frequency increases with age. A concern of surgeons performing the pedicle screw fixation surgery on patients suffering from osteoporosis is the probability of loosening or pullout failure of the screw during or after surgery.

To overcome the drawbacks of osteoporosis in pedicle screw fixations, several methods have been used. In one method increasing the major diameter Da of the screw leads to stronger engagement of the screw and bone. Another method is to inject a special kind of biocompatible cement through the screw in order to glue it to the surrounding bone. Even though this method has shown good performance in pullout tests, it poses several disadvantages which restricts its application. One disadvantage that the implanted pedicle screw with this method is that it is not removable after insertion.

Orthopedic fastener 200 is made of two portions, an apical section that functions as an Osteotome, and a coronal section that is similar to a standard pedicle screw implant, providing a fixation function. This design allows orthopedic fastener 200 to make its own osteotomy, fixate itself to the desired position as it is inserted into the bone.

The shape and the geometry of the apical portion of orthopedic fastener 200 is correlated to the shape and size of the threaded area. The upper section of apical portion is slightly larger than the diameter of the core of the implant of the lower thread, to allow smooth insertion and grip of bone by the first thread. As the implant is inserted forward, the core widens, to ensure mild better condensation of surrounding bone while the other threads ensure fixation into bone. The result is a stable implant even in soft bone conditions. Bone condensation is used in the art in clinical situations where bone density is considered insufficient to support a dental implant.

The osteotome area of orthopedic fastener 200 does not cut bone. The edgeless design of the osteotome area provides bone condensation. During implant placement, the curvature surface, combination of concave and convex geometries, mildly condenses surrounding bone laterally to provide better mechanical property of the surrounding hosting bone.

Orthopedic fastener 200 includes an implant body 202 having a proximal threaded region 204 having a thread 206 and a distal region 208 having a plurality of edgeless flutes 210. In one implementation thread 206 is a screw thread. Each edgeless flute 210 has a flute profile with a radius of curvature to densify a bone without grinding bone. The flute profile is similar to the flute profile discussed above herein with respect to implant 110. In one implementation each edgeless flute 210 has a flute profile having a radius of curvature greater than 0.1 mm along an entirety of the flute profile. However, other radius of curvatures are anticipated such as one of: greater than 0.05 mm; greater than 15 mm; and greater than 0.2 mm. In one implementation the radius of curvature along the entirety of the flute profile that contacts the bone is sufficient to density the bone but not cut or grind the bone. In one implementation each flute profile has a region of infinite curvature. In one implementation each edgeless flute extends less than 360 degrees about a longitudinal axis of the implant body. Distal region 208 has a tapered shape between a first distal diameter and a second distal diameter.

Proximal threaded region 204 of orthopedic fastener 200 includes a thread 206 having a minor diameter Db that is less than the first distal diameter Dc. Implant body 202 has a blunt tip 212 proximate a distal terminal end 214 of the implant body 202. The blunt tip 212 has an edgeless arcuate shape. Blunt tip 212 has a diameter Dd that is less than the diameter of the distal region 208. Orthopedic fastener 200 includes a proximal head portion 216 including a feature 220 to receive a screwdriver, Allen wrench, or any additional instruments such tulip and rod as is known in the art. Wherein the feature 220 includes a shaped recess.

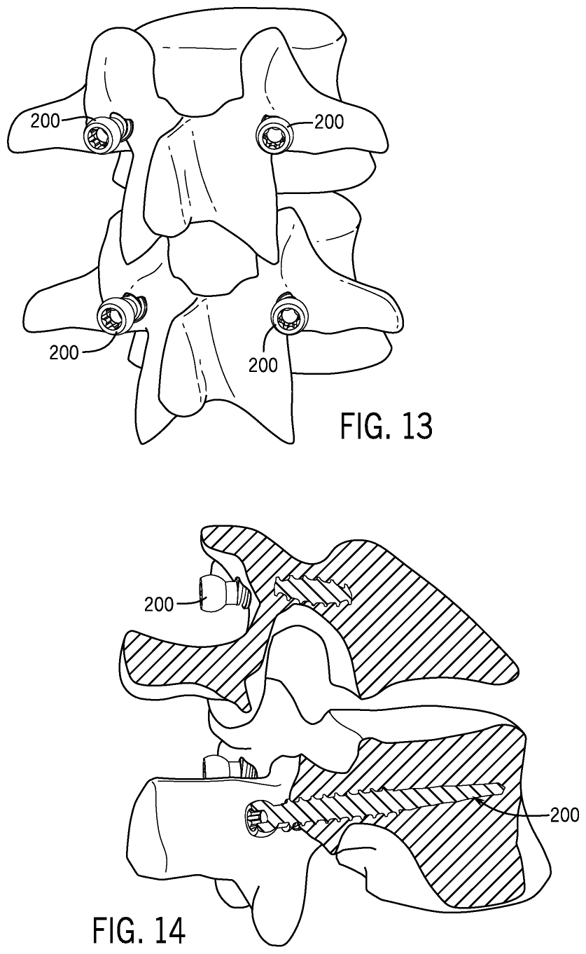

Referring to FIG. 13 and FIG. 14 orthopedic fastener 200 is placed into a bone in a single rotational insertion includes forming an osteotomy within a bone by a low speed rotation of orthopedic fastener 200 and a simultaneous insertion of the orthopedic fastener into the bone; densifying the bone surrounding the osteotomy during the rotational insertion of the orthopedic fastener into the bone; and securing a proximal threaded portion of the orthopedic fastener to a coronal portion of the osteotomy at an end portion of the rotational insertion of the orthopedic fastener into the bone.

In a further implementation forming an osteotomy and densifying the bone occur during rotational insertion of the orthopedic fastener along a first linear depth. Orthopedic fastener 200 includes a distal end having a blunt terminal tip 212. Densifying the bone is performed with a plurality of edgeless flutes 210 on distal region 208 of implant body 202. The osteotomy is created and enlarged during rotational insertion along a first linear depth. Each edgeless flute 210 extends less than 360 degrees about a longitudinal axis 218 of implant body 202.

In a further implementation each flute 210 includes a curved radius on a leading edge and a trailing edge of a convex portion of the flute profile. Wherein the convex portion contacts and densifies bone when the orthopedic fastener 200 is being inserted into the bone. Densifying the bone includes densifying the bone without cutting or grinding bone in any direction during rotational insertion of orthopedic fastener 200. Each edgeless flute 210 includes a portion having an infinite curvature.

Creating the osteotomy includes gradually enlarging the osteotomy as the orthopedic fastener is rotationally inserted into the bone by a rotary sliding embossing process. Securing the coronal threaded portion of the orthopedic fastener to a coronal portion of the osteotomy occurs during insertion of the orthopedic fastener to a second linear insertion depth.

A leading portion of the coronal threaded portion engages a wall of the osteotomy below an opening of the osteotomy. A major diameter of the proximal threaded portion has a diameter greater than the diameter of the opening of the osteotomy. Proximal portion 216 has a recess to receive a tool such as a screwdriver, an Allen wrench, or any additional instruments such as a tulip and rod. The speed of rotation of orthopedic fastener 200 is less than 50 revolutions per minute as the orthopedic fastener 200 is being rotationally inserted into the bone. In one implementation the speed of rotation of orthopedic fastener 200 is less between 10 and 25 revolutions per minute.

Distal region 208 is configured to widen an osteotomy in a bone is made of a bone integrated property material such as titanium or ceramic. In one implementation the entire orthopedic fastener 200 is formed from a bone integrated property material such as titanium or ceramic.

Provided below are non-limiting embodiments.

Illustrative embodiment 1. A method of placing a dental implant in a patient's jaw comprising: forming an osteotomy within a bone of a patient's jaw by a low speed rotation of a dental implant and simultaneous insertion of the dental implant into the bone; densifying a first portion of the bone radially and shifting a second portion of the bone forward with a non-cutting process to an indentation region of a tip region of the dental implant by a plurality of edgeless shaped flutes helically winding about an apical region of the dental implant during the low speed rotational insertion of the dental implant into the bone; wherein the indentation region covers more than 50% of a leading surface area at the tip region of the dental implant; and securing a coronal threaded portion of the dental implant to a coronal portion of the osteotomy at an end portion of the rotational insertion of the dental implant into the bone.

Illustrative embodiment 2. The method of illustrative embodiment 1, further including raising a Schneiderian membrane into a sinus cavity with a blunt tip during the low speed rotational insertion of the dental implant into the bone and shifting the second portion of the bone into the sinus cavity.

Illustrative embodiment 3. The method of illustrative embodiment 2, further including osteotomy preparation, penetrating a sinus floor, and peeling a portion of the Schneiderian membrane with the blunt tip, wherein forming the osteotomy, penetrating the sinus floor, peeling a portion of the Schneiderian membrane, and securing a coronal threaded portion to the coronal portion of the osteotomy are accomplished in a single continual rotational insertion of the dental implant.

Illustrative embodiment 4. The method of any one of illustrative embodiments 1-2, wherein forming the osteotomy is free of irrigation.

Illustrative embodiment 5. The method of any one of illustrative embodiments 1-2, wherein an apical end of the dental implant includes an arcuate blunt and edgeless terminal tip.

Illustrative embodiment 6. The method of any one of illustrative embodiments 1-2, wherein forming the osteotomy is created and enlarged during rotational insertion along a first linear depth.

Illustrative embodiment 7. The method of any one of illustrative embodiments 1-2, wherein each edgeless flute includes a curved radius on a leading edge and a trailing edge of the edgeless flute.

Illustrative embodiment 8. The method of any one of illustrative embodiments 1-2, wherein densifying the bone includes densifying the bone free of cutting or grinding the bone when the dental implant is rotated in both a first direction and in a second direction opposite the first direction.

Illustrative embodiment 9. The method of any one of illustrative embodiments 1-2, wherein the low speed of rotation is less than 50 revolutions per minute.

Illustrative embodiment 10. The method of any one of illustrative embodiments 1-2, wherein the low speed of rotation is between 10 and 25 revolutions per minute.

Illustrative embodiment 11. The method of any one of illustrative embodiments 1-2, wherein a region between the flutes and a terminal end of the tip region is free of threads and including a step of partially retracting the dental implant from the jaw during rotational insertion of the dental implant.

Illustrative embodiment 12. The method of any one of illustrative embodiments 1-2, wherein the coronal region is free of a shank extending from a free end of the coronal region in a direction away from an apical end of the dental implant and wherein the implant includes an abutment within an internal coronal portion of the dental implant.

Illustrative embodiment 13. The method of any one of illustrative embodiments 1-2, wherein the coronal threaded portion includes a thread helically extending about a coronal region of the dental implant in a first direction, and the flutes helically extending about the apical region in a second direction opposite the first direction.

Illustrative embodiment 14. The method of any one of illustrative embodiments 1-2, wherein each flute includes a portion having an infinite curvature.

Illustrative embodiment 15. The method of any one of illustrative embodiments 1-2, wherein creating the osteotomy includes gradually enlarging the osteotomy as the dental implant is rotationally inserted into the bone by a rotary sliding embossing process.

Illustrative embodiment 16. The method of any one of illustrative embodiments 1-2, wherein securing a coronal threaded portion of the dental implant to a coronal portion of the osteotomy occurs during insertion of the dental implant to a second linear insertion depth.

Illustrative embodiment 17. The method of any one of illustrative embodiments 1-2, wherein forming an osteotomy and densifying and shifting the bone while maintaining bone matrix occurs during slow speed rotational insertion of the dental implant along a first linear depth.

Illustrative embodiment 18. The method of any one of illustrative embodiments 1-2, wherein each edgeless flute extends less than 360 degrees about a longitudinal axis of the dental implant.

Illustrative embodiment 19. The method of any one of illustrative embodiments 1-2, wherein a leading portion of a minor diameter of the coronal threaded portion engages a wall of the osteotomy below a coronal opening of the osteotomy and wherein a major diameter of the coronal threaded portion is greater than the diameter of an opening of the osteotomy.

Illustrative embodiment 20. The method of any one of illustrative embodiments 1-2, wherein the coronal threaded portion includes a thread helically extending about a coronal region of the dental implant in a first direction, and the flutes helically extending about the apical region in a second direction opposite the first direction.

Illustrative embodiment 21. A self-osteotome dental implant comprising: an implant body having a coronal threaded region with a helical thread; and an apical region having a plurality of flutes defining raceways between adjacent flutes, wherein the flutes extend helically about the implant body in a direction opposite the direction of the helical thread, a tip region having a plurality of indentations adjacent to a terminal free end of the implant body in fluid communication with the raceways, the indentations covering more than 50% of a leading surface area at the tip region of the implant, wherein the region between the flutes and the apical terminal free end of the implant body is free of threads. In one implementation the region between the flutes and the apical terminal free end of the implant body is free of any constraint that would inhibit removal and insertion of the dental implant during rotational insertion of the dental implant into the jaw.

Illustrative embodiment 22. The self-osteotome dental implant of illustrative embodiment 21, wherein each edgeless flute has a flute profile with a radius of curvature to densify a bone without grinding bone.

Illustrative embodiment 23. The self-osteotome dental implant of illustrative embodiment 22, wherein each edgeless flute has a flute profile having a radius of curvature greater than 0.05 mm along an entirety of the flute profile.

Illustrative embodiment 24. The self-osteotome dental implant of illustrative embodiment 23, wherein each flute profile has a region of infinite curvature.

Illustrative embodiment 25. The self-osteotome dental implant of illustrative embodiment 21, wherein each edgeless flute extends less than 360 degrees about a longitudinal axis of the implant body.

Illustrative embodiment 26. The self-osteotome dental implant of illustrative embodiment 21, wherein the apical region has a tapered shape between a first apical diameter and a second apical diameter.

Illustrative embodiment 27. The self-osteotome dental implant of illustrative embodiment 26, wherein the coronal threaded region includes a thread having a leading edge with a minor diameter that is less than the first apical diameter.

Illustrative embodiment 28. The self-osteotome dental implant of illustrative embodiment 26, wherein a major diameter of the coronal thread is greater than the first apical diameter.

Illustrative embodiment 29. The self-osteotome dental implant of illustrative embodiment 21, wherein the implant has a blunt tip on an apical terminal end of the implant body.

Illustrative embodiment 30. The self-osteotome dental implant of illustrative embodiment 29, wherein the blunt tip has an edgeless arcuate shape greater than 0.1 mm.

Illustrative embodiment 31. The self-osteotome dental implant of illustrative embodiment 30, wherein the blunt tip has a diameter that is less than the diameter of the fluted region.

Illustrative embodiment 32. The self-osteotome dental implant of illustrative embodiment 21, further including a prosthesis located within an internal cavity having an opening at a coronal end of the implant body.

Illustrative embodiment 33. The self-osteotome dental implant of illustrative embodiment 21, wherein the apical region which widens the osteotome in a bone is made of bone integrated property material such as titanium or ceramic.

Illustrative embodiment 34. A method of placing an orthopedic fastener into a bone comprising: forming an osteotomy within a bone by a low speed rotation of an orthopedic fastener and a simultaneous insertion of the orthopedic fastener into the bone; densifying the bone surrounding the osteotomy during the rotational insertion of the orthopedic fastener into the bone; and securing a proximal threaded portion of the orthopedic fastener to a proximal portion of the osteotomy at an end portion of the rotational insertion of the orthopedic fastener into the bone.

Illustrative embodiment 35. The method of illustrative embodiment 34, wherein forming an osteotomy and densifying the bone occur during rotational insertion of the orthopedic fastener along a first linear depth.

Illustrative embodiment 36. The method of illustrative embodiment 35, wherein the orthopedic fastener includes a distal end having a blunt terminal tip.

Illustrative embodiment 37. The method of illustrative embodiment 35, wherein densifying the bone is performed with a plurality of edgeless flutes on a fluted region of the orthopedic fastener.

Illustrative embodiment 38. The method of illustrative embodiment 37, wherein the osteotomy is created and enlarged during rotational insertion along the first linear depth.

Illustrative embodiment 39. The method of illustrative embodiment 37, wherein each edgeless flute extends less than 360 degrees about a longitudinal axis of the orthopedic fastener.

Illustrative embodiment 40. The method of illustrative embodiment 37, wherein each flute includes a curved radius on a leading edge and a trailing edge.

Illustrative embodiment 41. The method of illustrative embodiment 40 wherein densifying the bone includes densifying the bone without cutting or grinding bone in any direction.

Illustrative embodiment 42. The method of illustrative embodiment 40, wherein each flute includes a portion having an infinite curvature.

Illustrative embodiment 43. The method of illustrative embodiment 37, wherein creating the osteotomy includes gradually enlarging the osteotomy as the orthopedic fastener is rotationally inserted into the bone by a rotary sliding embossing process.

Illustrative embodiment 44. The method of illustrative embodiment 34, wherein securing the proximal threaded portion of the orthopedic fastener to a proximal portion of the osteotomy occurs during insertion of the orthopedic fastener to a second linear insertion depth.

Illustrative embodiment 45. The method of illustrative embodiment 44, wherein a leading portion of a minor diameter of the proximal threaded portion engages a wall of the osteotomy below a proximal opening of the osteotomy.

Illustrative embodiment 46. The method of illustrative embodiment 45, wherein a major diameter of the proximal threaded portion has a diameter greater than the diameter of an opening of the osteotomy.

Illustrative embodiment 47. The method of illustrative embodiment 34, further including a proximal head portion to receive a screw driver or any additional instruments such as a tulip and rod.

Illustrative embodiment 48. The method of illustrative embodiment 34, wherein the speed of rotation is less than 50 revolutions per minute.

Illustrative embodiment 49. The method of illustrative embodiment 34, wherein the speed of rotation is between 10 and 25 revolutions per minute.

Illustrative embodiment 50. An orthopedic fastener comprising: an implant body having a proximal threaded region; and a distal region having a plurality of edgeless flutes.

Illustrative embodiment 51. The orthopedic fastener of illustrative embodiment 50, wherein each edgeless flute has a flute profile with a radius of curvature to densify a bone without grinding bone.

Illustrative embodiment 52. The orthopedic fastener of illustrative embodiment 51, wherein each edgeless flute has a flute profile having a radius of curvature greater than 0.05 mm along an entirety of the flute profile.

Illustrative embodiment 53. The orthopedic fastener of illustrative embodiment 52, wherein each flute profile has a region of infinite curvature.

Illustrative embodiment 54. The orthopedic fastener of illustrative embodiment 50, wherein each edgeless flute extends less than 360 degrees about a longitudinal axis of the implant body.

Illustrative embodiment 55. The orthopedic fastener of illustrative embodiment 50, wherein the distal region has a tapered shape between a first distal diameter and a second distal diameter.

Illustrative embodiment 56. The orthopedic fastener of illustrative embodiment 55, wherein the proximal threaded region includes a thread having a minor diameter that is less than the first distal diameter.

Illustrative embodiment 57. The orthopedic fastener of illustrative embodiment 50, wherein the implant body has a blunt tip on a distal terminal end of the implant body.

Illustrative embodiment 58. The orthopedic fastener of illustrative embodiment 57, wherein the blunt tip has an edgeless arcuate shape.

Illustrative embodiment 59. The orthopedic fastener of illustrative embodiment 57, wherein the blunt tip has a diameter that is less than the diameter of the distal region.

Illustrative embodiment 60. The orthopedic fastener of illustrative embodiment 50, further including in a proximal portion a feature to receive a screwdriver or any additional instruments such tulip and rod.

Illustrative embodiment 61. The orthopedic fastener of illustrative embodiment 50, wherein the distal region of the implant which widens an osteotomy in a bone is made of a bone integrated property material such as titanium or ceramic.

Illustrative embodiment 62. The method of illustrative embodiment 1, wherein forming an osteotomy includes forming an osteotomy to the coronal threaded portion.

Although the present disclosure has been described with reference to example embodiments, workers skilled in the art will recognize that changes may be made in form and detail without departing from the spirit and scope of the defined subject matter. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described is manifestly intended to be as broad as possible. For example, unless specifically otherwise noted, the definitions reciting a single particular element also encompass a plurality of such particular elements.

1-20. (canceled)

21. A self-osteotome dental implant comprising:
an implant body having a coronal threaded region with a helical thread; and
an apical region having a plurality of flutes defining raceways between adjacent flutes, wherein the flutes extend helically about the implant body in a direction opposite the direction of the helical thread,
a tip region having a plurality of indentations adjacent to a terminal free end of the implant body in fluid communication with the raceways,
wherein the region between the flutes and the apical terminal free end of the implant body is free of threads.

22. The self-osteotome dental implant of claim 21, wherein each edgeless flute has a flute profile with a radius of curvature to densify a bone without grinding bone.

23. The self-osteotome dental implant of claim 22, wherein each edgeless flute has a flute profile having a radius of curvature greater than 0.05 mm along an entirety of the flute profile.

24. The self-osteotome dental implant of claim 23, wherein each flute profile has a region of infinite curvature.

25. The self-osteotome dental implant of claim 21, wherein each edgeless flute extends less than 360 degrees about a longitudinal axis of the implant body.

26. The self-osteotome dental implant of claim 21, wherein the apical region has a tapered shape between a first apical diameter and a second apical diameter.

27. The self-osteotome dental implant of claim 26, wherein the coronal threaded region includes a thread having a leading edge with a minor diameter that is less than the first apical diameter.

28. The self-osteotome dental implant of claim 26, wherein a major diameter of the coronal thread is greater than the first apical diameter.

29. The self-osteotome dental implant of claim 21, wherein the implant has a blunt tip on an apical terminal end of the implant body.

30. The self-osteotome dental implant of claim 29, wherein the blunt tip has an edgeless arcuate shape greater than 0.1mm.

31. The self-osteotome dental implant of claim 30, wherein the blunt tip has a diameter that is less than the diameter of the fluted region.

32. The self-osteotome dental implant of claim 21, further including a prosthesis located within an internal cavity having an opening at a coronal end of the implant body.

33. The self-osteotome dental implant of claim 21, wherein the apical region which widens an osteotome in a bone is made of bone integrated property material such as titanium or ceramic.

34. The self-osteotome dental implant of claim 21, wherein the indentations cover more than 50% of a leading surface area at the tip region of the implant.

35. The self-osteotome dental implant of claim 29, wherein the indentations cover more than 50% of a leading surface area at the tip region of the implant.

36. A method of placing a dental implant in a patient's jaw comprising:
forming an osteotomy within a bone of a patient's jaw by a low speed rotation of a dental implant and simultaneous insertion of the dental implant into the bone;
densifying a first portion of the bone radially and shifting a second portion of the bone forward with a non-cutting process to an indentation region of a tip region of the dental implant by a plurality of edgeless shaped flutes helically winding about an apical region of the dental implant during the low speed rotational insertion of the dental implant into the bone; and
securing a coronal threaded portion of the dental implant to a coronal portion of the osteotomy at an end portion of the rotational insertion of the dental implant into the bone.

37. The method of claim 36, further including raising a Schneiderian membrane into a sinus cavity with a blunt tip during the low speed rotational insertion of the dental implant into the bone and shifting the second portion of the bone into the sinus cavity.

38. The method of claim 37, further including osteotomy preparation, penetrating a sinus floor, and peeling a portion of the Schneiderian membrane with the blunt tip, wherein forming the osteotomy, penetrating the sinus floor, peeling a portion of the Schneiderian membrane, and securing a coronal threaded portion to the coronal portion of the osteotomy are accomplished in a single continual rotational insertion of the dental implant.

39. The method of claim 36, wherein an apical end of the dental implant includes an arcuate blunt and edgeless terminal tip.

40. The method of claim 36, wherein the low speed of rotation is less than 50 revolutions per minute.

41-60. (canceled)
